SEQUENCE LISTING (2 sequences)

SEQ ID NO: 1 - DNA, 21 bp, Artificial sequence
  Primer
ttatcccagcagaaacaggaa

SEQ ID NO: 2 - DNA, 20 bp, Artificial sequence
  Primer
tgactttggggattgtaggg

(19) United States
(12) Patent Application Publication
(10) Pub. No.: US 2014/0206562 A1
(43) Pub. Date: Jul. 24, 2014

(54) FABRICATION AND USE OF A MICROFLUIDICS MULTITEMPERATURE FLEXIBLE REACTION DEVICE

(71) Applicant: QUANTUMDX GROUP LIMITED, PURLEY (GB)

(72) Inventors: JOHN EDWARD MCCORMACK, NORTH WARRANDYTE (AU); ELAINE HARRINGTON WARBURTON, PURLEY (GB); JONATHAN JAMES O'HALLORAN, UCKFIELD (GB); MATTHEW DANIEL SOLOMON, HUGHESDALE (AU); DAVID JAMES BRIGGS, LANCEFIELD (AU); MINDY LEE ANDRE, CROYDON (AU); MATTHIAS SCHUENEMANN, BRUNSWICK (AU)

(73) Assignee: QUANTUMDX GROUP LIMITED, PURLEY (GB)

(21) Appl. No.: 14/159,219

(22) Filed: Jan. 20, 2014

Related U.S. Application Data
(63) Continuation of application No. 13/990,764.
(60) Provisional application No. 61/418,272, filed on Nov. 30, 2010.

(51) Int. Cl. B01L 7/00; C12Q 1/68; C12M 1/00; G01N 33/53

(52) U.S. Cl. CPC B01L 7/525; G01N 33/5302; C12Q 1/686; C12Q 1/6869; C12M 47/06
USPC 506/9; 435/303.1; 422/69; 435/306.1; 436/501; 435/91.2; 435/287.2; 435/91.21; 435/6.12; 435/5; 435/6.11; 506/39; 156/242

(57) ABSTRACT

Fabrication of a microfluidic multi-temperature reaction device (MMR) and the design and fabrication of the equipment to drive various molecular biological methods on the device are provided. The device can be applicable, for example, to nucleic acid (DNA, RNA, cDNA, etc) amplification, cell lysis, reverse transcription and other enzymatic temperature sensitive and also temperature cycling reactions.

### CROSS REFERENCE TO RELATED APPLICATIONS

This application is a Continuation application of U.S. application Ser. No. 13/990,764, filed May 30, 2013, which is the national phase under 35 U.S.C. §371 of PCT International Application No. PCT/IB2011/003015 which has an International filing date of Nov. 30, 2011, designating the United States of America, which claims the benefit of U.S. Provisional Patent Application No. 61/418,272 filed on Nov. 30, 2010, the disclosures of which are hereby expressly incorporated by reference in their entirety and are hereby expressly made a portion of this application.

### REFERENCE TO SEQUENCE LISTING, TABLE, OR COMPUTER PROGRAM LISTING

The present application incorporates by reference the sequence listing submitted as an ASCII text file. The Sequence Listing is provided as a txt file, which is 787 bytes in size.

### BACKGROUND OF THE INVENTION

1. Field of the Invention

Various embodiments of the present disclosures generally relate to the fabrication of a microfluidic multi-temperature reaction device (MMR) and the design and fabrication of the equipment to drive the molecular biological methods on the device for; nucleic acid (DNA, RNA, cDNA, etc) amplification, cell lysis, reverse transcription and other enzymatic temperature sensitive and also temperature cycling reactions.

2. Description of the Related Art

Molecular biology is the study of biology at a molecular level. In one aspect, molecular biology may concern understanding the interactions between the various systems of a cell, including the interactions between DNA, RNA and proteins as well as learning about the interactions, mechanisms and dynamics of molecular species as well as their regulation.

### SUMMARY OF THE INVENTION

According to one aspect of the present invention, a portable device for conducting a biological reaction is provided. The device may comprise a cassette, and one or more temperature-controlling elements. The temperature-controlling elements may be configured to provide at least one temperature zone to the sample. The device may be portable or handheld. The cassette may be configured to move the sample. The cassette may comprise one or more channels that may be configured to move the sample through the at least one temperature zone. In some embodiments, the device may be configured to conduct a biological reaction selected from the group consisting of a polymerase chain reaction, a helicase-dependent amplification, a recombinase polymerase amplification, a reverse transcription polymerase chain reaction, a nucleic acids sequencing, a lysis reaction, an immunoassay, a metabolic assay, and a detection reaction. In some other embodiments, each of the one or more temperature-controlling elements may be individually controlled.

In some embodiments, the one or more temperature-controlling elements in the foregoing device may comprise heat conducting metals. In some other embodiments, the one or more temperature-controlling elements may comprise aluminum. In some alternative embodiments, the one or more temperature-controlling elements may comprise heat conducting polymers. In certain some embodiments, the one or more temperature-controlling elements may be heated by a Kapton heater. In some other embodiments, the one or more temperature-controlling elements may be heated by a peltier heater.

In some embodiments, a temperature of each of one or more temperature-controlling elements of the foregoing device may be monitored by a temperature sensor which may provide a feedback loop to allow a controlling electronics to maintain each of the temperature-controlling elements at a desired temperature. In some other embodiments, the one or more temperature-controlling elements may comprise three or more of individual temperature-controlling elements. In still some other embodiments the one or more temperature-controlling elements may be linearly arrayed. Alternatively, the one or more temperature-controlling elements may be arrayed in a non-linear arrangement.

According to certain embodiments, the one or more temperature-controlling elements of the foregoing device may be integrated into the portable device. Further, in some embodiments, the cassette may be selected from the group consisting of a macro-, micro-, nano- and pico-fluidic devices. In some other embodiments, the cassette may comprise a polycarbonate material. In still some other embodiments, the polycarbonate material may be selected from the group consisting of glass and PDMS.

In some alternative embodiments, a surface of the channels of the cassette may be configured to have a reduced binding to a sample and/or a reaction reagent. Further, in some other embodiments, the cassette may further comprise a reaction reagent. In still some other embodiments, the cassette may further comprise one or more selected from the group consisting of a sample receiving chamber, a sample collection chamber, and a reservoir chamber. In certain embodiments, the cassette may be a single flow-through macro-, micro-, nano-, or pico-fluidic device.

In still some alternative embodiments, when the device is used to conduct a nucleic acid amplification reaction, a temperature of each of the one or more temperature-controlling elements does not need to be changed more than once per reaction. Further, in some other embodiments, the cassette may be configured to conduct lysis of the sample and/or extraction of nucleic acids from the sample.

According to another aspect of the present invention, a method of conducting a biological reaction is provided. The method may comprise providing a sample to the cassette of the foregoing device, and providing at least one temperature zone to the sample by controlling a temperature of each of the one or more temperature-controlling elements. In some embodiments, the biological reaction conducted by the method may be selected from the group consisting of a polymerase chain reaction, a helicase-dependent amplification, a recombinase polymerase amplification, a reverse transcription polymerase chain reaction, a nucleotide sequencing, a lysis reaction, an immunoassay, a metabolic assay, and a detection reaction. Further, in some other embodiments, when the method is used for a nucleic acid amplification reaction, a temperature of each of said one or more temperature-controlling elements does not need to be changed more than once per reaction.

According to still another aspect of the present invention, a method of manufacturing a cassette used in the foregoing device is provided. The method may comprise molding two halves of the cassette, and assembling the molded two halves of the cassette. In some embodiments, the method may further comprise, prior or after the assembling, treating the cassette to reduce binding of a sample and/or a reaction reagent to a surface of one or more channels. In certain some embodiments, treatment of the cassette may comprise one or more of the following: flowing a substance through one or more channels of the cassette, depositing a hydrophilic or hydrophobic material on the surface of one or more channels of the cassette, coating the surface of one or more channels of the cassette with UV, and placing polymer brushes on the surface of one or more channels of the cassette. In some embodiments, the substance flowing through the channels of the cassette may be bovine serum and/or polymerase enzymes. In some other embodiments, the hydrophilic or hydrophobic material deposited on the surface may be fluorocarbons, Teflon, polyacrylates, and/or mixtures thereof. In still some other embodiments, the method may further comprise attaching an enzyme to the surface of one or more channels of the cassette.

### BRIEF DESCRIPTION OF THE DRAWINGS

FIG. 1 illustrates a microfluidic process used in a microfluidic thermal reactor cassette according to some embodiments of the present invention.

FIG. 2 illustrates an exploded view of an extraction cartridge according to some embodiments of the present invention.

FIG. 3 illustrates a device according to some embodiments of the present invention. In this particular illustration, a microfluidics thermal reactor cassette is placed and processed.

FIG. 4 illustrates an exploded view of a device according to some embodiments of the present invention. In this particular illustration, a microfluidics thermal reactor cassette is placed and processed.

FIG. 5 illustrates a heater block used in a device according to some embodiments of the present invention.

FIG. 6 illustrates a microfluidic thermal reactor cassette placed in a device according to some embodiments of the present invention.

FIG. 7 illustrates a diagram of a microfluidic channel in a microfluidic thermal reactor cassette according to some embodiments of the present invention.

FIG. 8 illustrates diagrams of a microfluidic channel in a microfluidic thermal reactor cassette according to some embodiments of the present invention.

FIG. 9 illustrates diagrams of a microfluidic channel in a microfluidic thermal reactor cassette according to some embodiments of the present invention.

FIG. 10a shows a gel picture illustrating the results of a PCR experiment performed according to some embodiments of the present invention. FIG. 10b shows the sequences of the primers used for the PCR experiment.

FIG. 11a shows another gel picture illustrating the results of a PCR experiment performed according to some embodiments of the present invention. FIG. 11b shows the sequences of the primers used for the PCR experiment.

FIG. 12 shows the results from a Reverse Transcription PCR experiment performed according to some embodiments of the present invention.

FIG. 13 illustrates a microfluidics cassette designed for handheld diagnostics according to some embodiments of the present invention.

FIG. 14 illustrates a microfluidics cassette design designed for handheld sequencing according to some embodiments.

FIG. 15 provides an illustration of a point of care device and microfluidics cassette according to some embodiments of the present invention.

### DETAILED DESCRIPTION OF THE PREFERRED EMBODIMENT

The present invention generally relates to the fabrication of a microfluidic multi-temperature reaction device (MMR) and the design and fabrication of the equipment to drive the molecular biological methods on the device for; nucleic acid (DNA, RNA, cDNA, etc) amplification, cell lysis, reverse transcription and other enzymatic temperature sensitive and also temperature cycling reactions. According to some aspect, the present invention may provide a low cost, low power consuming technology, to allow for thermal cycling, as required by a number of biological experimental procedures, such that the technology can be deployed in a portable, handheld device.

By understanding the interactions, mechanisms and dynamics between DNA, RNA and proteins and how they relate to defined clinical states, one may be able to use the analysis of them for clinical diagnosis. Therefore, molecular diagnostics may describe diagnosis by the study of clinically relevant markers at the molecular level.

DNA may be one of the targets of molecular diagnostics and may consist of a long polymer of units called nucleotides. The DNA polymers are long chains of single units, which together form molecules called nucleic acids. Nucleotides can be one of four subunits (adenine (A), cytosine (C), guanine (G) & thymine (T)) and, when in a polymer, they may carry the genetic information in the cell. DNA often comprises two long chains of nucleotides comprising the four different nucleotides bases (e.g. AGTCATCGTAGCT . . . etc) with a backbone of sugars and phosphate groups joined by ester bonds, twisted into a double helix and joined by hydrogen bonds between the complementary nucleotides (A hydrogen bonds to T and C to G in the opposite strand). The sequence of nucleotide bases along the backbone may determine individual hereditary characteristics.

The central dogma of molecular biology generally describes the normal flow of biological information: DNA can be replicated to DNA, the genetic information in DNA can be ‘transcribed’ into mRNA, and proteins can be translated from the information in mRNA, in a process called translation, in which protein subunits (amino acids) are brought close enough to bond, in order (as dictated by the sequence of the mRNA & therefore the DNA) by the binding of tRNA (each tRNA carries a specific amino acid dependant on its sequence) to the mRNA.

Thus clinical diagnostics may take advantage of some or all types of biological molecules, DNA, RNA and proteins. One way to analyze many of these molecules may be to prepare the sample and perform reactions with them. In some cases the sample may be prepared (DNA extracted, or sample centrifuged and diluted for some protein assays) and a reaction performed. Also in some other cases this reaction may need a defined temperature that might not always be room temperature and often it may need two or more temperatures.

To study the sequence and biology of DNA or RNA from a sample one may need to extract, or isolate, the nucleic acids from the clinical or biological sample. This may be presently performed by a number of methods in test tubes or multiwell plates, but these methods are likely to be replaced by more automated technologies, likely to be deployed in nano- or mico-fluidic cassettes/chips. Following extraction the target region of the DNA may be amplified to a level that is detectable using amplification techniques such as such as polymerase chain reaction (PCR), Helicase-dependent amplification (HDA), recombinase polymerase amplification (RPA). In the case of Amplifying DNA using the polymerase chain reaction (PCR) method, the sample and reaction mix may cycle through 3 different temperature zone approximately 25-35 times at least in some applications. Detection may then be performed, for example, by Hybridization (such as southern blotting, microarrays, expression arrays, etc) at a specific temperature, or by monitoring the accumulation of a fluorescent tag specific to the amplified target region of DNA. Alternatively, the detection may be performed by sequencing this amplified region of DNA.

These methodologies may be deployed in tubes or multi-well plates, may needsubstantial hands on operator time, multiple steps and large, bulky and expensive equipment. Some aspects of the present invention may provide a solution for parts of the molecular biological work flow, in a small, cheap and disposable microfluidics and versatile cassette/chip. When integrated with a sample preparation cassette/chip and a detection cassette/chip, it may provide a powerful, automated, cheap, simple and portable solution to the expensive molecular biology laboratory according to at least some embodiments.

In one aspect, the present application is to provide a low cost, low power consuming methodology to allow for thermal cycling, which may be involved in a number of biological experimental procedures (e.g. the thermal cycling for the polymerase chain reaction, PCR), such that the technology can be deployed in a portable, handheld device. Presently, thermal cycling for experiments such as PCR may need the reaction mix and sample to be heated and cooled as many as 40 times (for PCR) and this constant heating and cooling is considered extremely power consuming. Isothermal methodologies may have gained recent favor with point of care, portable devices as they may require the heating of the reaction to a single temperature and to hold the reaction at that temperature for the duration of the experiment. However, these isothermal methods may be limited in their scope (such as a limit to the multiplexing potential of isothermal DNA amplification methods, such as LAMP, RPA, HAD, etc, over PCR). Therefore, the inventors developed the technology, which is presented in at least some embodiments of the present invention, to facilitate the use of PCR and other protocols in hand held devices, without the requirement to heat and cool heater elements more than once per reaction.

The design of a microfluidics device and the design and fabrication of the equipment to drive the molecular biological methods on the device for; nucleic acid (DNA, RNA, cDNA, etc) amplification, cell lysis, reverse transcription and other enzymatic temperature sensitive and also temperature cycling reactions and the use of the device in running these reactions is disclosed in accordance with some embodiments of the present invention. The method may comprise: a design of the microfluidic cassette and the design and fabrication of the equipment to provide the fluidic flow and temperature zones. Furthermore, the method may comprise methods of use for the device, including PCR, RT-PCR, lysis and protein & metabolic assays all on the cassette and performs the reactions efficiently and to a standard that facilitates down stream use of the reaction products (i.e. in a colourmetric or fluorescent detection device, a nanopore, carbon nanotube, nanogap or nanowire biosensor).

One of the elements/features of the Microfluidics Multi-temperature Reaction device is an array of heating blocks and a methodology of passing a reaction mix or sample through temperature zones created by these arrays of heater blocks. The heater blocks can be formed of any heat conducting material and heated using any controlled method of heating the blocks. The temperatures may be modulated by a sensor that feedbacks temperature information to the heating module to ensure the blocks are kept at a desired temperature. For a reaction the heater blocks may be heated to their set temperature and remain at that temperature throughout the experiment, and the sample may be moved, flowed, shuttled, or other through temperature zones created by these heater blocks, to create the thermal conditions (e.g. such a thermal cycling required for PCR) for the reaction.

In one embodiment, the method for flowing a sample reaction mix through the different temperature zones, created by the array of heater blocks may be a single flow-through macro-, micro-, nano-, or pico-fluidic cassette, that consists of looping channels that may pass through the relevant temperature zones in the order required by the experiment.

Further additions to the single flow-through macro-, micro-, nano-, or pico-fluidic cassette may include a sample chamber and a collection chamber. Further embodiments may have on cassette reagent storage reservoirs that can contain reagents or water that can reconstitute lyophilized reagents deposited in the channels.

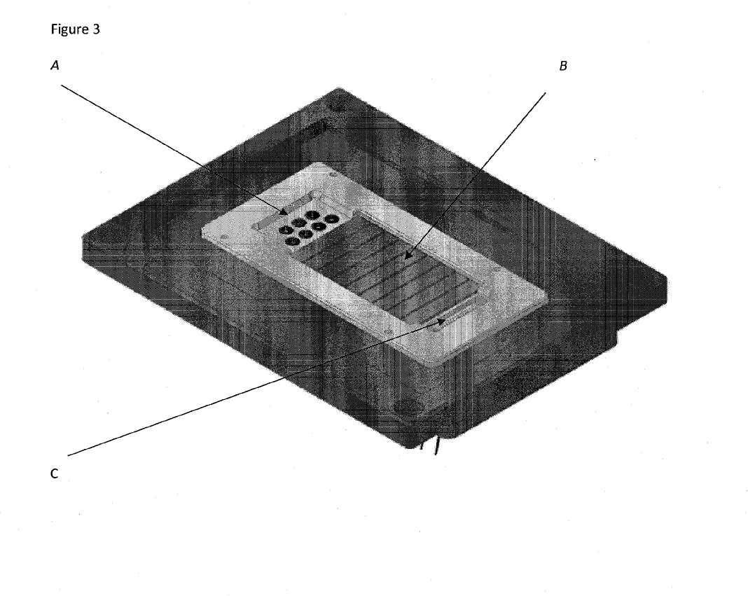

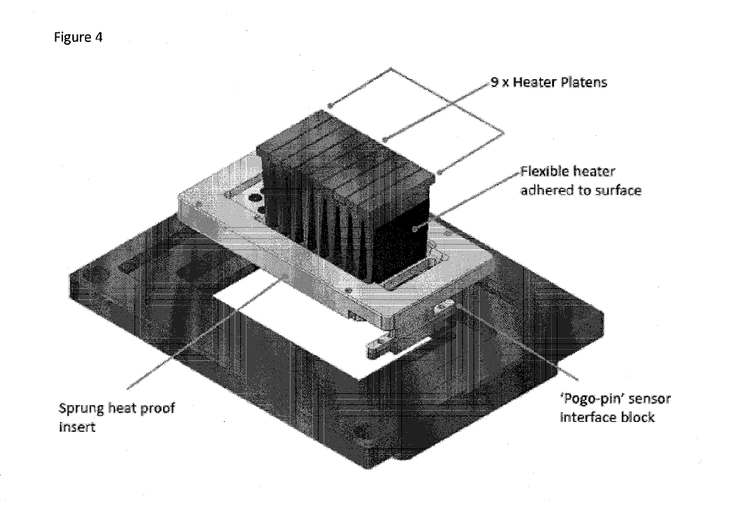

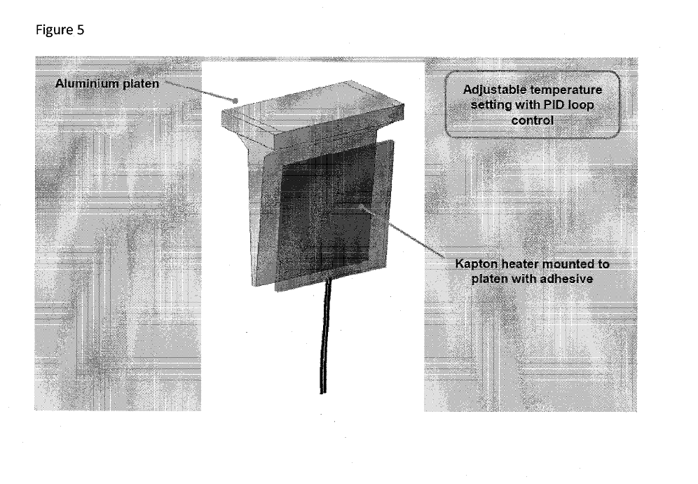

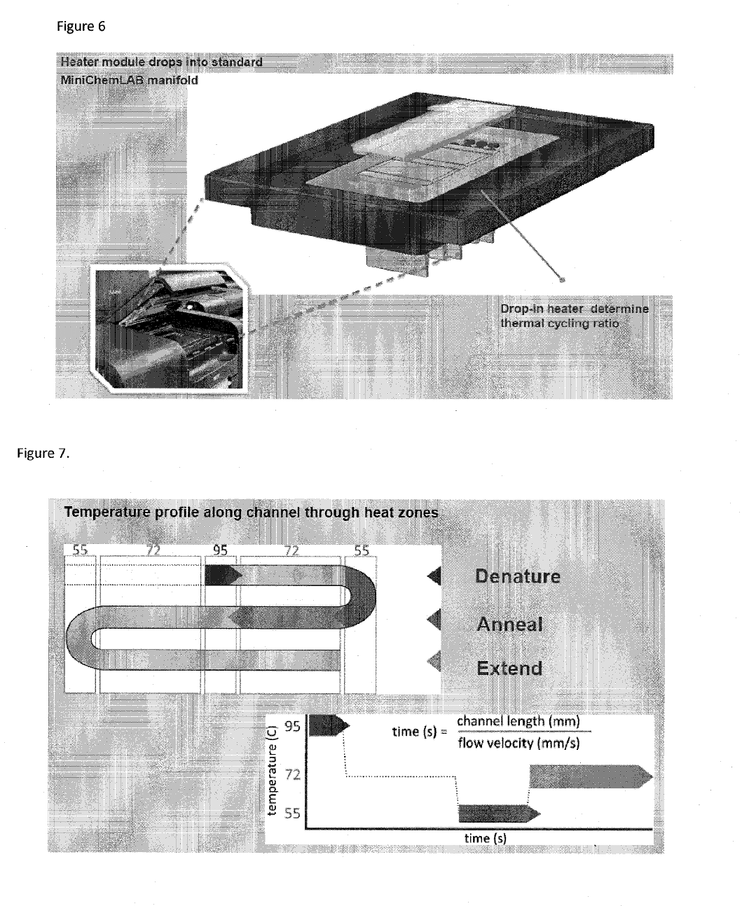

According to some embodiments, a microfluidics thermal reactor cassette may be placed and processed in the device. With respect to FIG. 3, for example, the microfluidics thermal reactor cassette may comprise A. Fluidic entry ports, B. Block heaters, and C. Pogo pin array, or another method of measuring a biosensor embedded in the cassette.

In some embodiments, the single flow-through macro-, micro-, nano-, or pico-fluidic microfluidics multi-temperature reaction device cassette can be fabricated by injection molding, or other process, two halves of a polycarbonate, or other material, shell, such that they create the desired looping lay out. Tooling for the injection molding process may be fabricated by electroforming a polymer master designed to incorporate functional elements for the correct functioning of the final device. All components may be thoroughly washed and dried before assembly. The two halves of the cassette may be thermally bonded with a thin layer sealing film over the channel structures by lamination or hydraulic press. Cartridge components may be firmly bonded together using a pressure based system (e.g. lamination, hydraulic press). Other bonding methodologies may be employed in other embodiments.

In some embodiments of the device, the cassette may be injection moulded or milled from a polycarbonate, or other biocompatible plastic material. In other embodiments the cassette may be be moulded or milled from glass or PDMS or another biocompatible material.

One of the problems with devices such as these flow-through cassettes may be the issue of high surface area to volume ratio. This may provide for a large hydrophobic surface area that might cause biologically active reaction constituents, such as enzymes, to bind and denature on the surfaces, thus rendering them inactive and deleteriously affecting the experiment. Therefore, according to some embodiments, prevention or reduction of enzymes (e.g. DNA polymerases, Reverse Transcriptases, etc) from denaturing on the surface may be preferred.

In some embodiments of the single flow-through macro-, micro-, nano-, or pico-fluidic microfluidics multi-temperature reaction device cassette, the surface of the channels may be treated such as to prevent or reduce absorption and adsorption into and onto the material of any reaction or sample constituents. Such surface treatment may comprise methods including but not limited to; flowing a sacrificial substance through the channel, thereby reducing loss of material, treating the surface with biological material such as bovine serum, polymerase enzymes or other such materials, or chemically treating the surface to prevent loss. Treatments may include but are not limited to the placement of materials that may create a hydrophilic or hydrophobic surface to allow a smoother flow. In some other embodiments fluorocarbons and similar materials (Teflon, as an example would act as a hydrophobic harrier, or polyacrylates) may be deposited onto the surface of the channels. Other methodologies such as UV coatings and polymer brushes that are chemically grown off the surface may also be included in this invention. In yet another embodiment it may be that the material that the cartridge is fabricated from may be chosen or adapted in its design and material make-up, to prevent or reduce loss of material onto or into the surface.

Surface chemistries such as those outlined above, may be used to prevent the enzymes from denaturing on the surface of the microfluidic channels. In some embodiments this treatment may actually enhance the performance of the enzyme or allow further stability of the enzyme.

In other embodiments the enzyme may be attached to the surface using chemical or biological linkers to prevent or reduce denaturation or loss of material. Such linkers may include but are not limited to di-sulphide linkers, bis-amine linkers, silane chemistries, peptide recognition moieties, histidine tagging linkers, ion recognition moieties as well as biological species that may show an affinity to the surface and/or the enzyme itself. In some embodiments the enzymes (Taq polymerase as an example) may have specifically been adapted to bind to surface species or be coupled with enzymes with such properties. In yet another embodiment it may be that the material that the cartridge is fabricated from may be chosen or adapted in its design and material make-up, to enzyme denaturation or promote further stability or performance of the enzyme.

In some embodiments the placement of enzymes may be done specifically in certain regions of the microfluidic channel to provide optimum conditions for the amplification to take place. Placement of the enzyme may be carried out using printing techniques or the surface may have been treated in specific areas to maximise the enzyme present in these areas. In some embodiments this placement may be carried out by either enhancing the enzyme affinity to certain areas within the channels or by blocking areas on the surface that do not require material to be placed onto it. Examples of such techniques are outlined above, and can be used to promote or demote adhesion to the surface in these areas. Printing techniques may be used to provide more accurate deposition of material and minimise waste, or may also be used to carry out in situ methodologies for attachment of material to the surface.

### EXAMPLES

The followings are some illustrative and non-limiting examples of some embodiments of the present disclosures.

### Example 1

### DNA Sequencing

By arraying biosensors (such as FETs, nanowire FETs, Carbon nanotubes, LED and other miniature fluorescent detectors, etc) throughout the microfluidics channels it may sense changes at each length of the channel. If one or more DNA molecules (of the same sequence) is elongated through the channel it may sense different bases at different positions by using the sequencing by synthesis as described in U.S. 61/094,006, or by using one of the pyrosequencing methods used by illumina and Roche. The temperature control may help with denaturing the DNA and with the polymerization of the DNA.

### Example 2

### PCR

Many molecular diagnostic methodologies, Cepheid (Smart Cycler), Light Cyeler (Roche), BeadXpress & Eco Real-Time PCR system (Illumina), 7500 Real-Time PCR System (ABI), GeneChip system (Affymetrix), cycle reaction mixes containing DNA templates, primers, dNTPs, polymerase and MgCl₂ to perform a PCR reaction to amplify specific regions of DNA to a level in which they can be detected. However all of the technologies to date employ heaters (usually peltier heaters) that ramp up and down the temperatures. According to some aspects of the present invention, this is inappropriate for a portable hand held device to be deployed in the field due to the energy wastage, costs and difficulty in cooling in hot ambient temperatures.

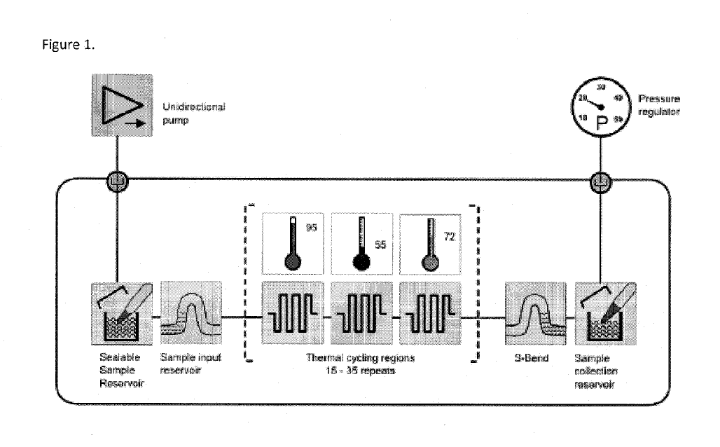

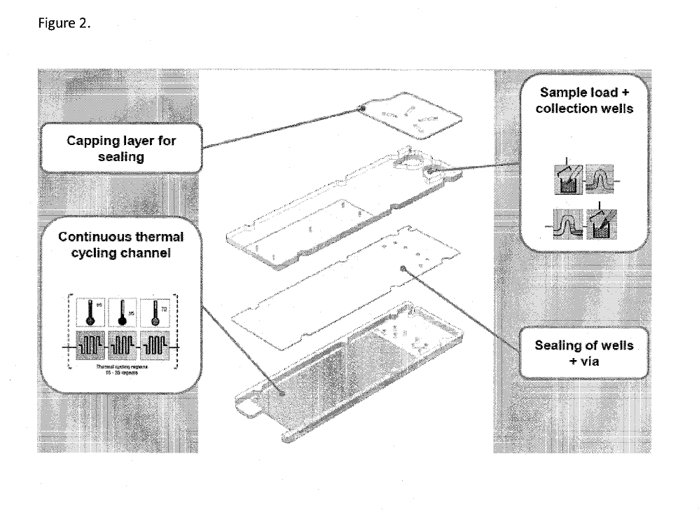

With respect to FIG. 1, which depicts some illustrative embodiments, the specific design is to perform a PCR (polymerase chain reaction) experiment, as it illustrates three different temperature zones (in this case 95° C., 55° C. and 72° C.). Due to the ability to set the different heat blocks off-cassette to different temperatures, there can be numerous different configurations and temperatures for different applications. For instance, all the heat blocks can be set to a single temperature, such as 68° C. for a reverse transcription reaction, or room temperature/37° C. for immunoassays. Other configurations can see two temperatures, for instance 0° C. and 90° C. for freeze thaw cycles, or more temperatures for more complex experiments.

An alternative embodiment is illustrated in FIG. 7. In this embodiment, the first heating block (left handside) is at 55° C. The next three are set as 72° C., with the next block set at 95° C. The next three blocks are set at 72° C. again and the final block on the right is set as 55° C. This is a configuration for PCR, allowing for the denaturation of the DNA at 95° C., the annealing of the primers to the template DNA at 55° C. and the extension at 72° C. Note that there is an extra extension phase that is not usually included in traditional PCR, however this is a function of the flow through method and does not deleteriously affect the PCR reaction efficiency. This is therefore a ratio of 1:3:2 (denature, anneal & extend) due to only one heat block at 95° C., three at 72° C. and finally the bend is at 55° C. and is therefore considered two, due to the time the reaction mix spends in the temperature zone.

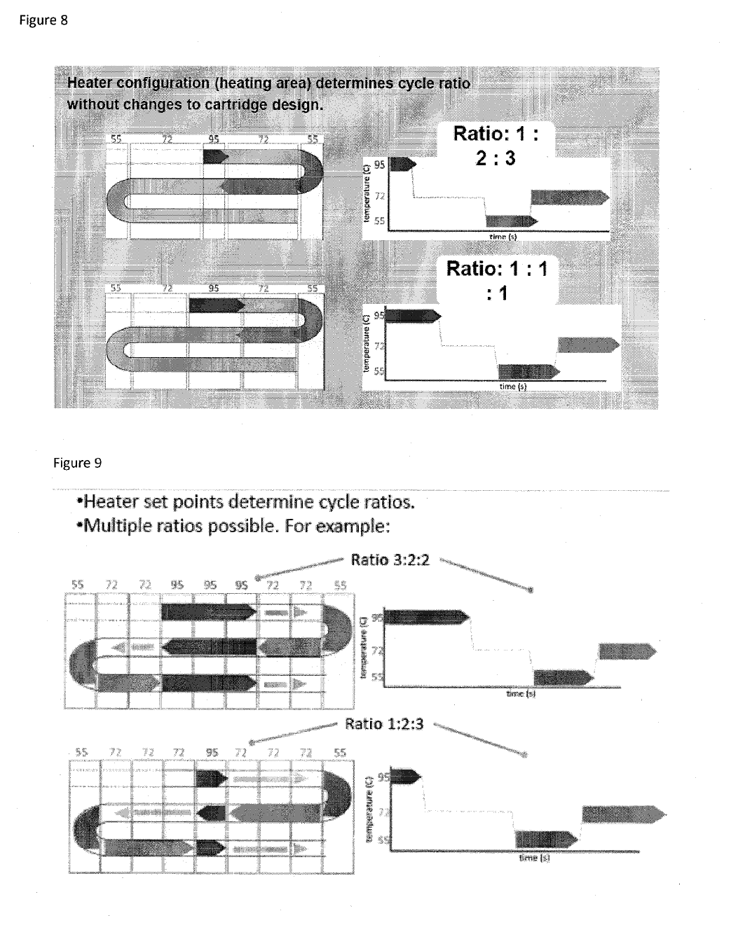

Further alternative embodiments are illustrated in FIG. 8. The diagrams in the figure illustrate different configurations that allow for the device to be flexible and cater for many different thermal programs, especially when the fluid flow rate is also altered to provide further flexibility.

With reference to FIG. 9, it illustrate further different configurations that allow for the device to be flexible and cater for many different thermal programs, especially when the fluid flow rate is also altered to provide further flexibility.

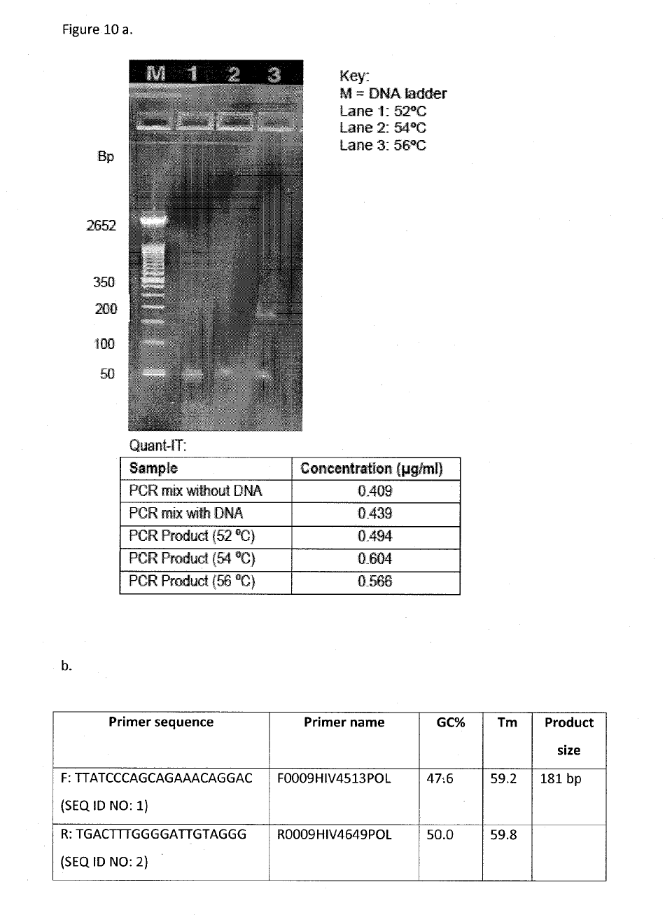

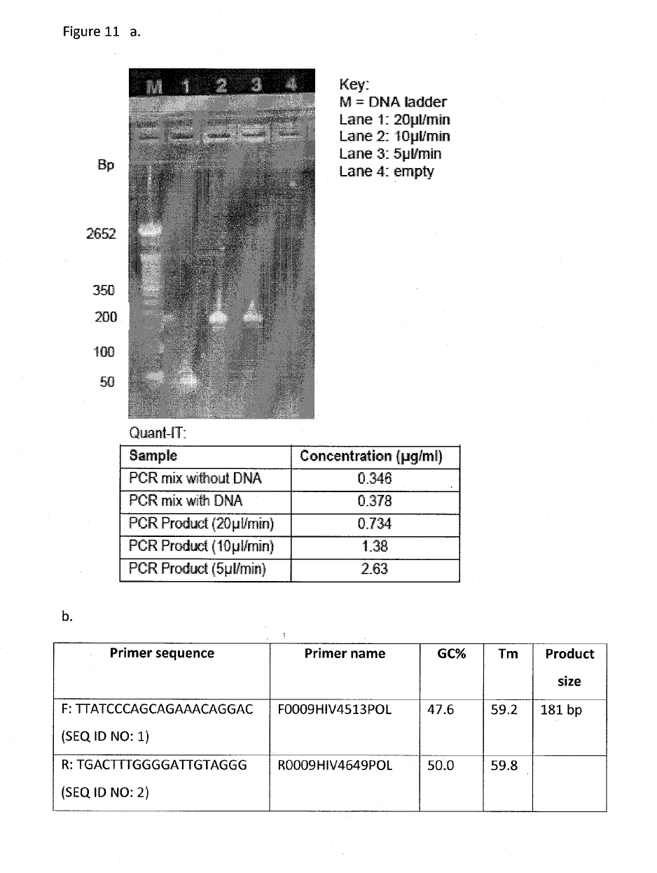

FIGS. 10 and 11 demonstrate successful applications of certain embodiments of the present invention for the PCR amplification of the pol region of the HIV gene. The gel pictures show the results of a PCR experiment wherein a region of the HIV pol gene is amplified (the target is positive control DNA plasmid insert), performed in the microfluidic thermal reactor cassette over the heating blocks. Three different fluidic flow rates were tested with the resultant eluates quantified with the Quant-IT system.

### Example 3

### RT-PCR

In another example the specific target molecule to be analysed may be RNA and may need a reverse transcription PCR experiment to be performed in the microfluidic thermal reactor cassette.

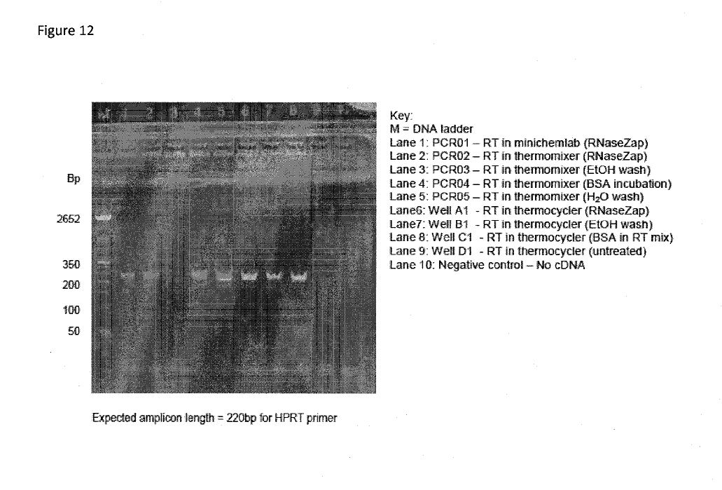

FIG. 12 demonstrates a reverse transcription PCR experiment result obtained according to some embodiments of the invention. The reaction was run in the microfluidic thermal reactor cassette, with a cDNA library produced from whole blood as the template. The reaction amplified a region of the house keeping gene HPRT.

### Example 4

### Handheld Sequencing and Diagnostic Device

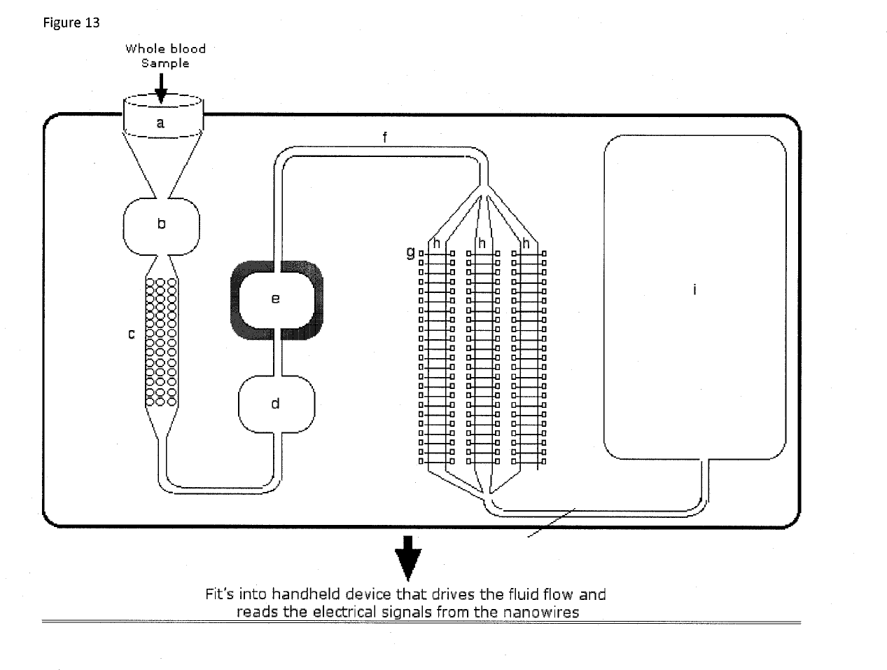

In some embodiment, specific target DNA sequences may be extracted in a microfluidics channel that may lead to further downstream processes in a single flow-through microfluidics cassette wherein the DNA is sequenced (FIG. 13). In some embodiments, all the reagents required for the lysis and extraction of DNA from samples can be stored in a microfluidics channel, each wash solution and lysis buffer, can be separated by an air bubble, or another method of separating the reagents, or the reagents can be stored in blister packs, lyophilized or other methods for storing reagents within microchannels.

In some embodiment, small specific regions of target viral, bacterial or genomic DNA can be extracted for sequencing and therefore be diagnostic for the presence or absence of a specific virus, bacteria, or genetic sequence (such as a SNP), as well as provide value-added information on genetic type, mutations (known or unknown), drug resistance status, etc.

At least some embodiments used in connection with the present disclosures lend itself to handheld sequencing as it may not require bulky and equipment required to extract the DNA prior to the sequencing reaction.

In one embodiment, a probe sequence can be immobilized on a sensitive detection nanostructure (in this case a nanowire) and the template ssDNA molecule to be sequenced can hybridize to the probe sequence and the probe sequence can act as a primer for the sequencing by synthesis reaction. In another embodiment the template ssDNA molecule can be immobilized to the sensitive detection nanostructure and can be primed for sequencing with a free primer oligonucleotide.

An illustrative embodiment is presented in FIG. 13. In this particular example, Q-POC disposable cassette may comprise; a. Sample reception. b. Sample lysis. Project IPR may be sought, in partnership with nexttec (Germany) for the specific lysis buffer/conditions. c. Sample preparation may be done, for example, by a method of packing nexttec's sorbant filter into a microfluidics channel in some embodiments. d. Concentration of the DNA may be done, for example, by DNA extraction in some embodiments. Reconstitution of lyophilized reagents (if using dry reagents) PCR reagents will also occur in this chamber, or mixing or wet reagents. e. Thermal cycling. f. Microfluidics. g. Nanowires, or other biosensors arrayed after or within some embodiments. h. Electronics are used to link the signal from the biosensors to the reader device. i. Waste is simply an empty microfluidics reservoir.

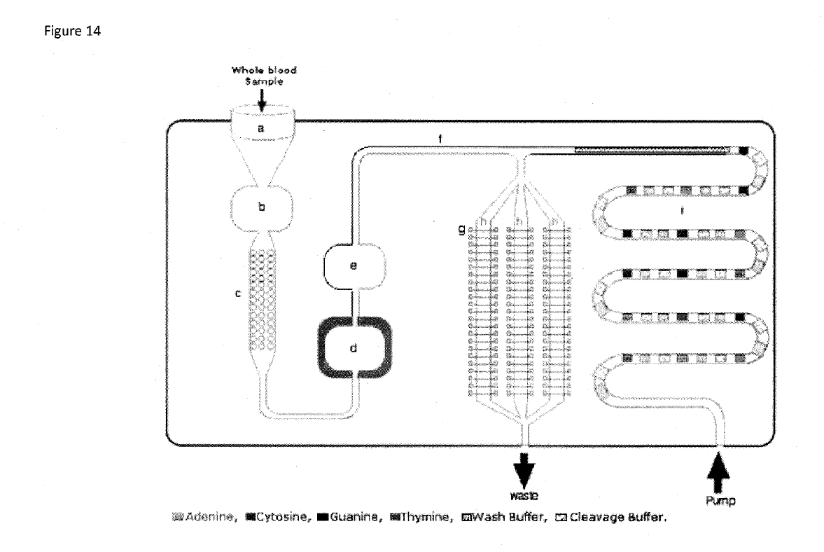

Another illustrative embodiment is presented in FIG. 14. In this particular example, the device may comprise: a. Sample Reception—This element may act as a barrier for the sample to escape and can yet be able to accept samples, much like the rubber may top on blood Vacutainers. h. Lysis Chamber—This illustrates a relatively simple microreactor, chamber which comprises a lysis reagent to break up the cells and to release genomic DNA. This section might also resemble a filter to remove blood cells if the target nucleotide polymer can be free in the blood serum. c. Nucleic Acid Sample Preparation—The nucleotide polymer fraction of the sample can be isolated and extracted from the rest of the sample constituents (proteins, carbohydrates, lipids, etc). This can be achieved by some methods well known to those skilled in the art. For instance, this maco-fluidic chamber might contain Nexttec's filter technology. d. Amplification of the Target Nucleotide Polymer—This section may amplify the target nucleotide polymer, using the polymerase chain reaction, which may employ heating elements or other well known strategies of cycling a reaction mix through the different temperatures required for PCR, to perform the thermal cycling required, or isothermal amplification methods (such as LAMB, RPA, etc), which may not require heating of the sample. Sample Processing—This might be required at least in some embodiments to concentrate the nucleic acids, or remove ‘over-hang’ nucleotide chains that might cause background signal, prior to sequencing. f. General Microfluidics—This describes the size of the channels, fluid flow, valves and control, materials and valves used in some embodiments. g. Metal connects—These connect the sensitive detection nanostructures (in this case, nanowires) to the detector device in some embodiments. h. Sensitive Detection Nanostructure Arrays—The microfluidics channel can be tightly arrayed sensitive detection nanostructures (such as nanowires, or carbon nanotubes). Two methods of positioning DNA in the channel can be employed; 1. tight channels may allow long stretches of DNA to uncoil, migrate & stretch down the channels which may allow for long read lengths if necessary, and 2. tiling probe/primers can be spotted on to nanowire clusters and short multiple parallel sequencing reactions performed throughout the channels. i. This weaving microfluidic channel can be filled with reagents in some embodiments, separated by air bubbles. As this microfluidics channel can be pumped, or a tiny actuator moves the reagents along, the sequence of the reagents in the microfluidics channel can run the sequencing by synthesis reaction. Alternatively, this method of reagent storage can be replaced with reservoirs or blister packs and also lyophilized reagents that are reconstituted by the reaction solution itself.

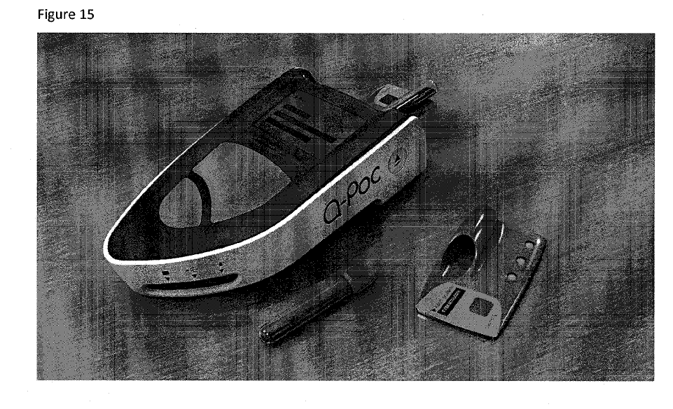

With reference to FIG. 15, which illustrates another embodiment, the device may include a DNA extraction and fractionation device within the cassette.

### Example 5

### Lysis

In another example the experiment can be a lysis step. This may be performed using a high salt buffer to break open the cell walls at room temperature in some embodiments. However, other methodologies can be used such as:

Freeze/Thaw Cycles.

The device according to some embodiments of the present invention may be suited to heating and cooling the sample rapidly, thereby creating the conditions required for rapid freeze thaw cycling.

Enzymatic Lysis.

The use of enzymes in lysis is well documented; however this method of lysis has not yet been performed well in microfluidics. The device according to some embodiments of the present invention may perform an enzymatic lysis as it may quickly bring a reaction to the temperature required by the enzymes and also, due to the altered dynamics within microchannels, compare to tube based systems, is able to speed up the lysis step.

### Example 6

### Immunoassay

As an immunoassay is a biochemical test that measures, usually using an antibody, the presence or concentration of a substance in solutions that frequently contain a complex mixture of substances, the optimum reaction temperature may be dictated by the antibody-antigen binding profile. Therefore in some cases the reaction may be maintained at a single temperature, or alternatively at series of temperatures especially when multiple antibodies are used. This therefore may make the device extremely versatile as it may be able to provide for 9 different binding temperatures in one reaction according to at least some embodiments.

Detection devices, such as colourmetric or fluorescent detection devices (such as an LED), a nanopore, nanogap, or carbon nanotube, and nanowire FET biosensors arrayed like sleepers on a train track along the microfluidic channel, can be built in the device according to at least some embodiments.

What is claimed is:

1. A device for conducting a biological reaction comprising:
a cassette that is configured to receive a sample; and
one or more temperature-controlling elements, wherein said one or more temperature-controlling elements is configured to provide at least one temperature zone to the sample;
wherein the cassette comprises one or more channels that are configured to move the sample through said at least one temperature zone; and
the device is portable or handheld.

2. The device according to claim 1, wherein the device is configured to conduct a biological reaction selected from the group consisting of a polymerase chain reaction, a helicase-dependent amplification, a recombinase polymerase amplification, a reverse transcription polymerase chain reaction, a nucleic acids sequencing, a lysis reaction, an immunoassay, a metabolic assay, and a detection reaction.

3. The device according to claim 1, wherein each of said one or more temperature-controlling elements is individually controlled.

4. The device according to claim 1, wherein said one or more temperature-controlling elements comprises heat conducting metals.

5. The device according to claim 1, wherein said one or more temperature-controlling elements comprises aluminum.

6. The device according to claim 1, wherein said one or more temperature-controlling elements comprises heat conducting polymers

7. The device according to claim 1, wherein said one or more temperature-controlling elements are heated by a Kapton heater.

8. The device according to claim 1, wherein said one or more temperature-controlling elements are heated by a pettier heater.

9. The device according to claim 1, wherein a temperature of each of one or more temperature-controlling elements is monitored by a temperature sensor which provides a feedback loop to allow a controlling electronics to maintain each of the temperature-controlling elements at a desired temperature.

10. The device according to claim 1, wherein said one or more temperature-controlling elements comprises three or more of individual temperature-controlling elements

11. The device according to claim 1, wherein said one or more temperature-controlling elements are linearly arrayed.

12. The device according to claim 1, wherein said one or more temperature-controlling elements are arrayed in a non-linear arrangement.

13. The device according to claim 1, wherein said one or more temperature-controlling elements are integrated into the portable device.

14. The device according to claim 1, where the cassette is selected from the group consisting of a macro-, micro-, nano- and pico-fluidic devices.

15. The device according to claim 14, wherein the cassette comprises a polycarbonate material.

16. The device according to claim 15, wherein the polycarbonate material is ed from the group consisting of glass and PDMS.

17. The device according to claim 14, wherein a surface of the channels of the cassette is configured to have a reduced binding to a sample and/or a reaction reagent.

18. The device according to 1, wherein the cassette further comprises a reaction reagent.

19. The device according to claim 1, wherein the cassette further comprises one or more selected from the group consisting of a sample receiving chamber, a sample collection chamber, and a reservoir chamber.

20. The device according to claim 1, wherein the cassette is a single flow-through macro-, micro-, nano-, or pico-fluidic device.

21. The device according to claim 1, when the device is used to conduct a nucleic acid amplification reaction, a temperature of each of said one or more temperature-controlling elements does not need to be changed more than once per reaction.

22. The device according to claim 1, wherein the cassette is configured to conduct lysis of the sample and/or extraction of nucleic acids from the sample.

23. A method of conducting a biological reaction comprising:
providing a sample to the cassette of the device according to claim 1;
providing at least one temperature zone to the sample by controlling a temperature of each of said one or more temperature-controlling elements.

24. The method according to claim 23, wherein the biological reaction is selected from the group consisting of a polymerase chain reaction, a helicase-dependent amplification, a recombinase polymerase amplification, a reverse transcription polymerase chain reaction, a nucleotide sequencing, a lysis reaction, an immunoassay, a metabolic assay, and a detection reaction.

25. The method according to claim 23, when the method is used for a nucleic acid amplification reaction, a temperature of each of said one or more temperature-controlling elements does not need to be changed more than once per reaction.

26. A method of manufacturing a cassette used in the device according to claim 1 comprising:
molding two halves of the cassette; and
assembling the molded two halves of the cassette.

27. The method according to claim 26, wherein the method further comprises:
prior or after said assembling, treating the cassette to reduce binding of a sample and/or a reaction reagent to a surface of one or more channels.

28. The method according to claim 27, wherein said treating comprises one or more of the following:
flowing a substance through one or more channels of the cassette, wherein said substance is selected from the group consisting of bovine serum and polymerase enzymes;
depositing a hydrophilic or hydrophobic material on the surface of one or more channels of the cassette, wherein said hydrophilic or hydrophobic material is selected from the group consisting of fluorocarbons, Teflon, and polyacrylates;
coating the surface of one or more channels of the cassette with UV; and
placing polymer brushes on the surface of one or more channels of the cassette.

29. The method according to claim 26, wherein the method further comprises:
attaching an enzyme to the surface of one or more channels of the cassette.
